(19) United States
(12) Patent Application Publication
(10) Pub. No.: US 2008/0177387 A1
(43) Pub. Date: Jul. 24, 2008

(54) Implants and Related Devices for Monitoring Bony Fusion

(75) Inventors: Dianna Parimore, Arlington, TN (US); Scott Hutton, Memphis, TN (US); Anthony J. Melkent, Memphis, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/555,339

(22) Filed: Nov. 1, 2006

(51) Int. Cl. A61F 2/44

(52) U.S. Cl. 623/17.16

(57) ABSTRACT

A medical implant for supporting skeletal structures is disclosed. The implant includes an expandable central portion formed of a first material that at least partially inhibits the monitoring of bone in-growth using a medical diagnostic technique. The implant further includes end caps for mating with the central portion and the skeletal structure. The end caps are made of a second material different than the first material that inhibits the monitoring of bone in-growth using the medical diagnostic technique to a lesser degree than the first material. In another aspect, a polymer end member for use with a metallic implant for supporting a skeletal structure is disclosed. The polymer end member facilitates the monitoring of fusion or bone in-growth using fluoroscopy. In another aspect, a spinal implant is disclosed. The implant includes a central portion made of a first material and an end cap made of a different material.

### TECHNICAL FIELD

Embodiments of the present disclosure relate generally to the field of replacing portions of the human structural anatomy with medical implants, and more particularly relate to implants and methods of replacing skeletal structures, such as one or more vertebrae or long bones.

### BACKGROUND

Characteristics of implantable-grade or medical-grade polymers-such as biocompatibility, strength, flexibility, wear resistance, and radiolucency-make them especially suitable for use in some medical device applications, such as spinal implants. In some aspects, medical-grade polymers can be used in combination with other materials, such as metals, to enhance the performance or desired characteristics of the implant. Although existing implants and methods have been generally adequate for their intended purposes, they have not been entirely satisfactory in all respects.

### SUMMARY

A spinal implant for positioning between a superior vertebra and an inferior vertebra is disclosed. In one embodiment, the spinal implant includes a central portion having a superior section, an inferior section, and a longitudinal axis extending therebetween. The central portion is made of a first material. The implant also includes a superior end cap having an inferior surface for engagement with the superior section of the central portion and a superior surface for engagement with the superior vertebra. The superior end cap is made of a second material different than the first material.

In a second embodiment, an expandable medical implant for supporting skeletal structures is provided. The implant includes an expandable central portion having a first end section, an opposing second end section, and a longitudinal axis extending therebetween. The central portion is formed of a first material that at least partially inhibits the monitoring of bone in-growth using a medical diagnostic technique. The implant also includes a first end cap having a first mating surface for mating with the first end section of the central portion and a first engagement surface for engagement with a first portion of the skeletal structure. The first end cap is made of a second material different than the first material, the second material inhibiting the monitoring of bone in-growth using the medical diagnostic technique to a lesser degree than the first material. The implant also includes a second end cap having a second mating surface for mating with the second end section of the central portion and a second engagement surface for engagement with a second portion of the skeletal structure. The second end cap is made of the second material.

In another embodiment, an end member for use with a metallic implant for supporting a skeletal structure is provided. The end member includes a body portion having a thickness. The end member also includes an implant engagement surface extending from the body portion for securely engaging the metallic implant. The end member also includes a skeletal engagement surface extending from the body portion opposite the implant engagement surface, the skeletal engagement surface for securely engaging the skeletal structure and promoting bony in-growth between the skeletal structure and the end member. The end member is formed of a polymer.

Additional and alternative features, advantages, uses, and embodiments are set forth in or will be apparent from the following description, drawings, and claims.

### BRIEF DESCRIPTION OF THE DRAWINGS

FIG. 1 is an elevation view of a segment of a lumbar spine.

FIG. 2 is a perspective view of an expandable implant according to one embodiment of the present disclosure.

FIG. 3 is side cross-sectional view of the implant of FIG. 2.

FIG. 4 is an enlarged view of a portion of the implant illustrated in FIG. 3.

FIG. 5 is an exploded view of the implant of FIG. 2.

FIG. 6 is a side view of a portion of the implant of FIG. 2, but showing an alternative embodiment.

FIG. 7 is a top view of the portion of the implant of FIG. 6.

FIG. 8 is a bottom view of the portion of the implant of FIG. 6.

FIG. 9 is a side view of a portion of the implant of FIG. 2, but showing an alternative embodiment.

FIG. 10 is a top view of the portion of the implant of FIG. 9.

FIG. 11 is a bottom view of the portion of the implant of FIG. 9.

FIG. 12 is a perspective, partially-exploded view of an implant according to one embodiment of the present disclosure.

FIG. 13 is a perspective bottom view of the implant of FIG. 12.

### DESCRIPTION

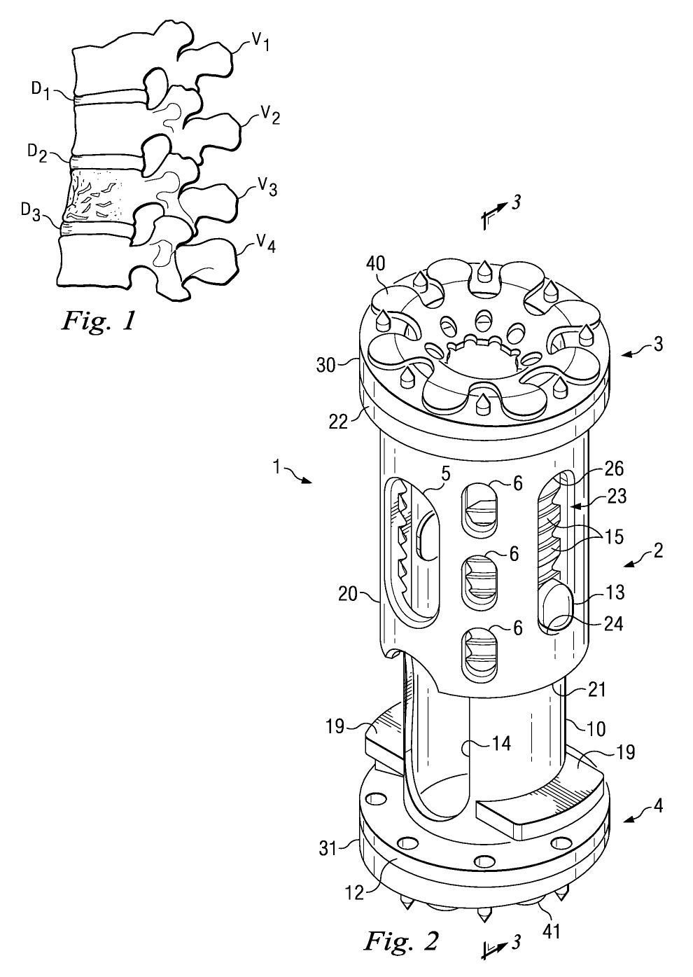

It is sometimes necessary to remove one or more vertebrae, or a portion of the vertebrae, from the human spine in response to various pathologies. For example, one or more of the vertebrae may become damaged as a result of tumor growth, or may become damaged by a traumatic or other event. Removal, or excision, of a vertebra may be referred to as a vertebrectomy. Excision of a generally anterior portion, or vertebral body, of the vertebra may be referred to as a corpectomy. An implant is usually placed between the remaining vertebrae to provide structural support for the spine as a part of a corpectomy. FIG. 1 illustrates four vertebrae, V1-V4 of a typical lumbar spine and three spinal discs, D1-D3. As illustrated, V3 is a damaged vertebra and all or a part of V3 could be removed to help stabilize the spine. If removed along with spinal discs D2 and D3, an implant may be placed between vertebrae V2 and V4. All or part of more than one vertebrae may be damaged and require removal and replacement in some circumstances. Most commonly, the implant inserted between the vertebrae is designed to facilitate fusion between remaining vertebrae. Sometimes the implant is designed to replace the function of the excised vertebra and discs.

Many implants are suitable for use in a corpectomy procedure. One class of implants is sized to directly replace the vertebra or vertebrae that are being replaced. Another class of implants is inserted into the body in a collapsed state and then expanded once properly positioned. Expandable implants can be advantageous because they allow for a smaller incision when properly positioning the implant. Additionally, expandable implants can assist with restoring proper loading to the anatomy and achieving more secure fixation of the implant. Expandable implants can also be useful outside of the spinal column in replacing long bones or portions of appendages such as the legs and arms, or a rib or other bone that is generally longer than it is wide. Examples include, but are not limited to, a femur, tibia, fibula, humerus, radius, ulna, phalanges, clavicle, and any of the ribs. Further, both expandable and non-expandable implants can be useful within the intramedullary canal of long bones.

Implants that include insertion and expansion mechanisms that are narrowly configured also provide clinical advantages. In some circumstances, it is desirable to have vertebral endplate contacting surfaces that effectively spread loading across the vertebral endplates. Some implants include a mechanism for securely locking the implant in desired positions, and in some situations, also for collapsing the implant. Further, fusion implants with an uninterrupted opening extending between their ends can also be advantageous because they allow for vascularization and bone growth through the entire implant.

Regardless of the various features an implant may or may not have, the implant is secured between the remaining bone structure or vertebrae. Often the ends of the implant are fixedly secured to the vertebrae. In some embodiments each end of the implant engages with the vertebrae via an end cap or an end piece. The end caps can include various features, such as projections, to facilitate engagement with the vertebrae. Further, the portions of the end cap that engage the vertebra can be treated to encourage bone in-growth. For example, engagement surfaces of the end cap can be chemically-etched, machined, sprayed, layered, fused, coated, or textured in a manner or with a material that facilitates the growth and attachment of bone.

Further, in some embodiments all or a portion of the interior and/or periphery of the implant is packed with a suitable osteogenic material or therapeutic composition. Osteogenic materials include, without limitation, autograft, allograft, xenograft, demineralized bone, synthetic and natural bone graft substitutes, such as bioceramics and polymers, and osteoinductive factors. A separate carrier to hold materials within the device may also be used. These carriers may include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenic compositions may include an effective amount of a bone morphogenetic protein (BMP), transforming growth factor β1, insulin-like growth factor, platelet-derived growth factor, fibroblast growth factor, LIM mineralization protein (LMP), and combinations thereof or other therapeutic or infection resistant agents, separately or held within a suitable carrier material. The implant can be packed prior to insertion, after insertion, or a combination of before and after.

It is often desirable to monitor the bone in-growth between the implant and the bone structure using medical diagnostic equipment, such as fluoroscopy, ultrasound, magnetic resonance, computed tomography, positron emission technology, or other known or future diagnostic techniques. In particular, it is often desirable to monitor the bone in-growth and fusion at the ends of the implant where the implant end caps and vertebrae meet. However, the implant itself can interfere with monitoring the progress of bone in-growth. For example, where the implant including the end caps are formed of a metal or other radiopaque material, the radiopaque material can prevent or severely impair the ability to monitor bone in-growth using x-ray or fluoroscopy. On the other hand, the use of radiopaque materials is desirable in some embodiments due to other physical characteristics of the material, such as strength, elasticity, or otherwise.

In one aspect, the present disclosure teaches an implant having a central portion formed of a radiopaque material and end caps formed of a radiolucent material, such that the bone in-growth and/or fusion between the vertebrae and implant adjacent the ends of the implant can be monitored using x-ray or fluoroscopy. More generally, the present disclosure teaches an implant having a central portion made of one material and at least one end portion made of a different material and connected to the central portion such that bone in-growth between the bone and implant adjacent the end portion can be monitored using medical diagnostic equipment.

The materials for the central portion and end caps can be selected based on a specific type of diagnostic equipment to be used. For example, in the case of fluoroscopy the central portion can be formed from a material that is more radiopaque than the end cap material or, in other words, the end caps can be formed from a material that is more radiolucent than the central portion material. In other embodiments, the material for the central portion can reflect or absorb the energy emitted by the diagnostic equipment while the material for the end caps allows the energy to transmit through. Examples of possible energy forms utilized by the diagnostic equipment include, but are not limited to acoustic, light or laser, x-rays, ultra-sonic, positron emissions, and other energy forms. In this manner the central portion material is substantially reflective and/or absorbs the energy, while the end cap material is substantially transmissive to the energy to facilitate monitoring of the bone in-growth at the end cap-to-bone structure interface. In other embodiments, the central portion material may be substantially transmissive, while the end cap material is substantially reflective and/or absorbative to facilitate monitoring of the bone in-growth at the end cap-to-bone structure interface.

In at least one aspect, the implant is a corpectomy device and, in some embodiments is expandable. In some embodiments, the end caps are modular such that the central portion can be used with a variety of end caps of different shapes, sizes, and/or materials and/or the end caps can be used with a variety of central portions of different shapes, sizes, and/or materials. The central portion and end caps may be formed from various suitable biocompatible material including metals such as cobalt-chromium alloys, titanium alloys, nickel titanium alloys, or stainless steel alloys. Ceramic materials such as aluminum oxide or alumina, zirconium oxide or zirconia, compact of particulate diamond, or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); or cross-linked UHMWPE. Further, as described above the central portion and end caps can each be formed of different materials, permitting metal on metal, metal on ceramic, metal on polymer, ceramic on ceramic, ceramic on polymer, or polymer on polymer constructions. In one particular embodiment, the central portion is formed from a metal, such as titanium, and the end caps are formed of a polymer, such as PEEK.

For the purpose of promoting a greater understanding of the principles of the disclosure, reference will now be made to the particular embodiments, or examples, illustrated in the drawings and specific language will be used to describe the embodiments. It will nevertheless be understood that no limitation of the scope of the disclosure is intended. Any alterations and further modifications of the described embodiments, and any further applications of the principles of the disclosure as described herein are contemplated as would normally occur to one skilled in the art to which the disclosure relates.

FIGS. 2-5 illustrate an expandable medical implant 1 for supporting skeletal structures. Generally, the medical implant 1 comprises a first tubular member 10, a second tubular member 20, two end caps 30 and 31, and two shoes 40 and 41. As described below, the first and second tubular members 10, 20 form a central portion 2, end cap 30 and shoe 40 form an end member 3, and end cap 31 and shoe 41 form an end member 4. In one embodiment, the central portion 2 is formed of titanium and the end members 3 and 4 are formed of PEEK.

In the illustrated embodiment, the medical implant 1 includes the first tubular member 10 with a connection end 11 and opposite first skeletal interface end 12, and the second tubular member 20 with a connection end 21 configured to engage with the connection end 11 of the first tubular member 10. The second tubular member 20 has an opposite second skeletal interface end 22. A key pin 13 is fixed to the first tubular member 10 and positioned in a slot 23 in the second tubular member 20 such that the key pin 13 guides translation between the first tubular member 10 and the second tubular member 20. The embodiment shown includes a medial aperture 5 through which bone growth material may be packed and through which bone growth may occur. Additionally, the medial aperture 5 is an aid in radiographic assessment when the implant 1 is made from a material that is not radiolucent. Openings 6 are also useful for packing of bone growth material, and provide channels through which bone growth may occur.

The term tubular as used herein includes generally cylindrical members as are illustrated in FIG. 2, but may also include other enclosed or partially enclosed cross-sectional shapes. By way of example and without limitation, tubular includes fully or partially, cylindrical, elliptical, rectangular, square, triangular, semi-circular, polygonal, and other cross-sectional shapes of these general types.

The illustrated key pin 13 guides the translation of the first and second tubular members 10, 20 and provides torsional stability between the tubular members 10, 20. In addition, as shown in FIG. 2, the key pin 13 provides a positive stop to the expansion of the medical implant 1 by limiting the travel of the second tubular member 20 with interference between the key pin 13 and the bottom 24 of the slot 23. Similarly, the key pin 13 provides a positive stop to the contraction of the medical implant 1 by limiting the travel of the second tubular member 20 with an interference between the key pin 13 and the top 26 of the slot 23. The key pin 13 also provides a connection interface between an insertion instrument and the first tubular member 10.

As shown in the illustrated embodiment, the first tubular member 10 fits within the second tubular member 20. However, in other embodiments, the first tubular member may be of greater diameter than the second tubular member with the connection between the two members being reversed in orientation. Alternatively, the first and second tubular members may be of approximately the same size, but have legs that exist coplanarly or within the same tubular geometry with the legs of the other.

As shown in FIGS. 2, 3, and 5, the first tubular member 10 includes a relief cut 14 to facilitate portions of the first tubular member 10 flexing away from the second tubular member 20 to permit translation between the first and second tubular members. The flexing may be induced by pulling the first tubular member 10 away from the second tubular member 20 to expand the medical implant 1. Referring now to FIG. 4, pulling the first tubular member 10 down while pulling the second tubular member 20 up causes the inclined first flank 17 of the first protrusions, or first set of teeth 15, to press against the second flank 27 of the second protrusions, or second set of teeth 25. Because the second tubular member 20 has a continuous cross-section, it has a relatively stronger lateral resistance than the first tubular member 10 with its relief cut 14. Therefore, the force induced between the first and second flanks, 17, 27, causes the first tubular member 10 to flex away from the second tubular member 20. In other embodiments, a relief cut in the second tubular member 20 and a continuous shape in the first tubular member 10 could cause flexing of the second tubular member rather than the first. The degree and direction of flexing can be controlled by the use of different materials, various degrees of relief cutting, different cross-sectional shapes, and the shapes of the teeth or protrusions employed, among other factors. The force required for various degrees of flexing of the members is proportional to the force required to expand the implant. Therefore, the force required to expand the implant may be maintained within a desirable range by controlling the factors detailed above.

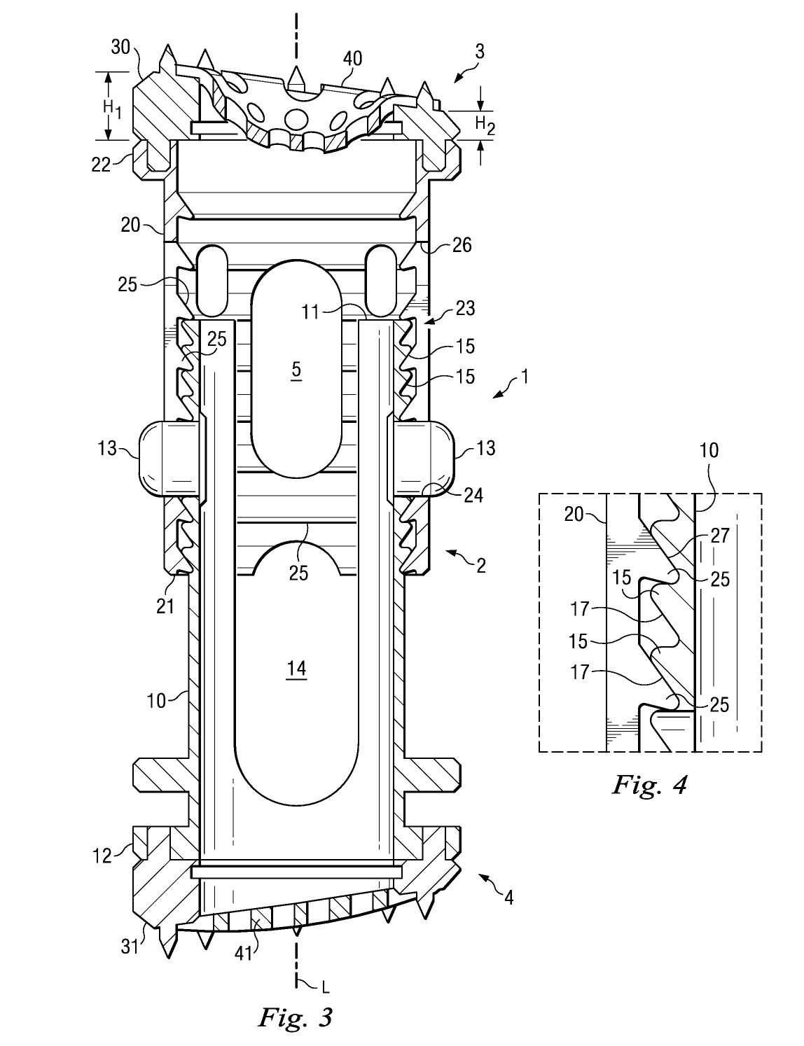

As best illustrated in FIGS. 3 and 4, the first tubular member 10 includes a set of first teeth 15, or more generally, protrusions, wherein the rows of teeth are adjacent to one another. The second tubular member 20 includes a set of second teeth 25, or more generally, protrusions, wherein the rows of teeth are not adjacent to one another. As shown, every other row of the set of second teeth 25 has been removed. However, in other embodiments, every third or fourth or some other number of rows may contain teeth, or the tooth pattern may repeat in some non-uniform fashion. If the sets of teeth were threads instead, a similar effect could be achieved by widening the pitch of the threads on one of the tubular members.

The first set of teeth 15 interdigitate with every other one of the teeth of the set of second teeth 25. This or other varied spacings may be advantageous. As noted above, the force required to expand the implant is proportional to the number of sets of teeth that are in contact while the tubular members 10, 20 are being translated. However, if teeth on both tubular members 10, 20 are spaced apart at greater distances, the number of increments to which the implant may be adjusted is decreased. By maintaining the frequency of the rows of the first set of teeth 15 and increasing frequency of the second set of teeth 25, the force required to expand the implant is reduced, but the number of discrete points of adjustment is not reduced. In some embodiments, the increased frequency of teeth could be maintained on the second tubular member 20 while the spacing is increased on the first tubular member 10.

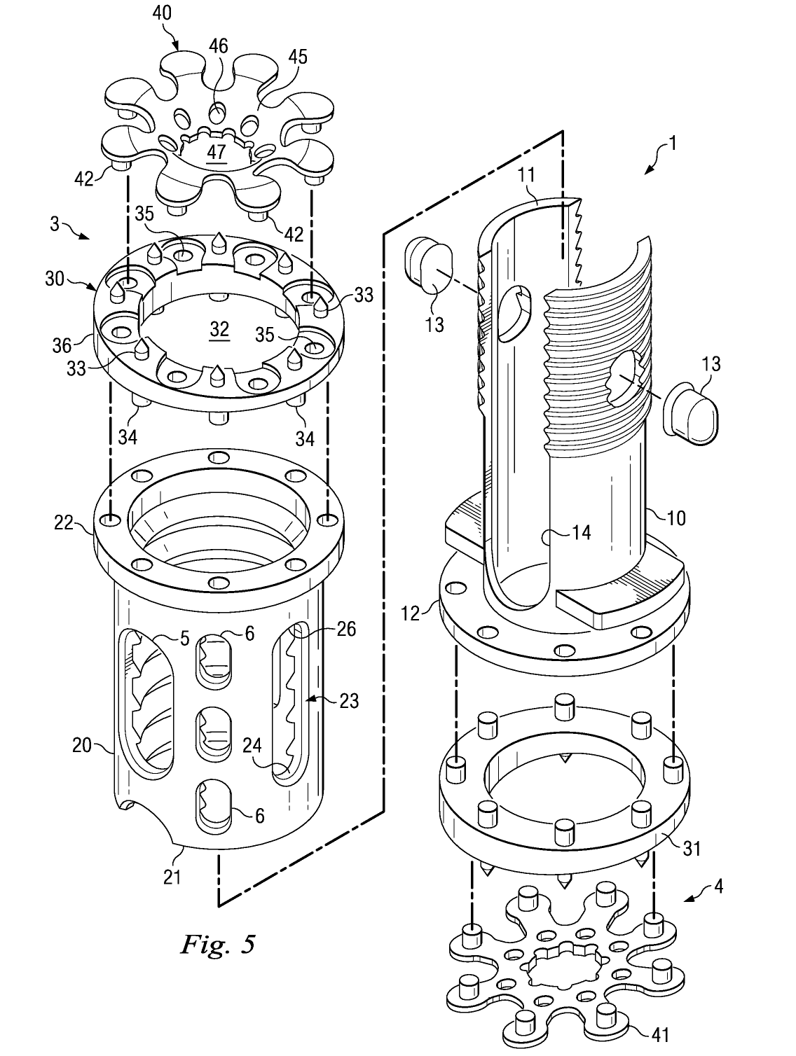

Referring now to FIGS. 5-8, the end member 3 includes end cap 30 and shoe 40 that mate with an end of the central portion 2 and provide connection to the skeletal structure, such as the vertebrae. End member 3 will now be described in detail. In some embodiments, end member 4 is substantially similar to end member 3 and, therefore, will not be described in detail. However, in other embodiments end member 4 (including end cap 31 and shoe 41) includes additional features, less features, or is otherwise different from end member 3 (including end cap 30 and shoe 40).

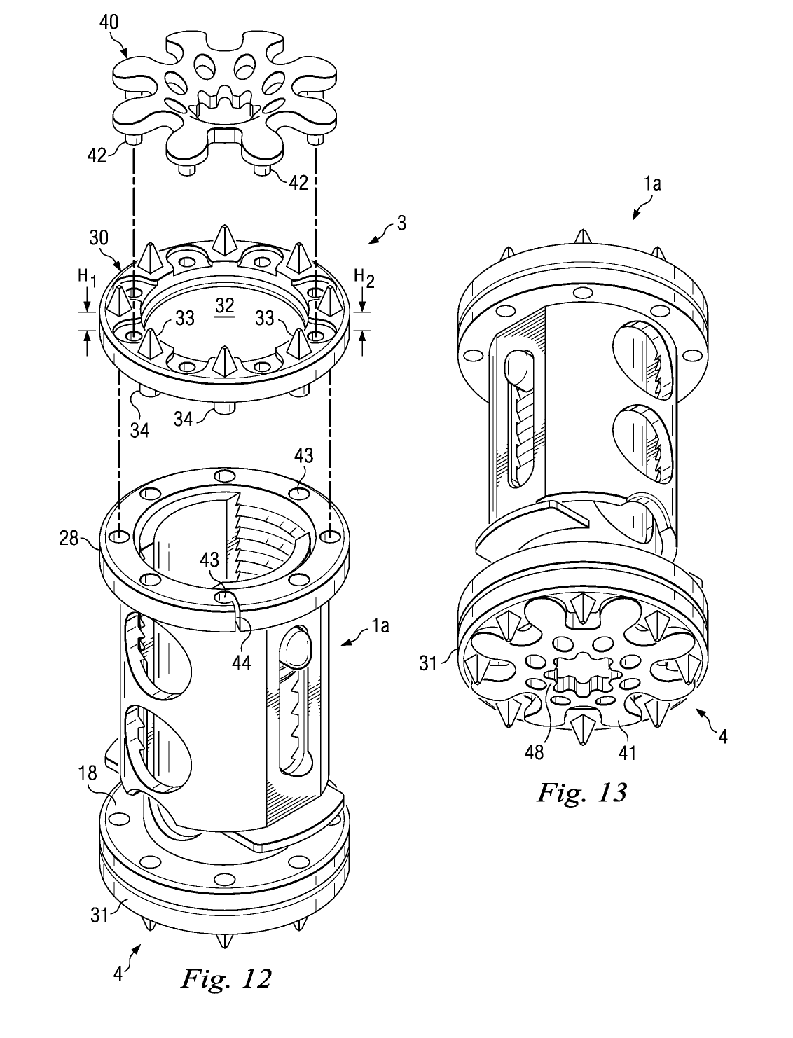

As shown in FIG. 5, the end member 3 is a separate component of the implant 1 that mates with the central portion 2 of the implant. In other embodiments, the end member 3 is integrated with the central portion 2 of the implant. The end member 3 may vary in thickness from H₁ to H₂, as shown in FIG. 3, such that placement of the end member 3 on the central portion 2 creates an interface with the bone structure that is not parallel to a longitudinal axis L extending along the length of the implant 1. This non-parallel configuration may enable the medical implant 1 to match the natural angles of a spinal curvature. For example, in much of the cervical and lumbar regions of the spine, the natural curvature is a lordotic angle. In much of the thoracic region of the spine, the natural curvature is a kyphotic angle. The variance in height between H₁ and H₂ can be selected to correspond to the desired angle based on the bone structure that the implant will interface with. As shown in FIG. 12, in other embodiments such as in implant la, the end member 3 in total and the end cap 30 may be of a uniform thickness such that H₁ and H₂ are approximately equal.

In some embodiments, the heights H₁ and H₂ or the thickness of the end cap 30 is in the range of 0.5 mm to 10 mm. The actual thickness of the end cap 30 can be tailored to match the resolution of the diagnostic equipment used to monitor fusion or bone in-growth. That is, the greater the resolution of the imaging, the smaller the thickness of the end cap 30 needs to be. However, the thickness can be substantially greater than necessary for monitoring fusion.

Referring again to FIGS. 5-8, the end cap 30 includes a number of surface irregularities that may aid connection or interface with the skeletal structure. In the current embodiment, the surface irregularities illustrated are spikes 33 that are sharp to penetrate the skeletal structure. In other embodiments, the surface irregularities may be raked or straight teeth that tend to bite into the skeletal structures to resist expulsion in particular directions, such as, for example, to resist expulsion opposite to the path of insertion. The surface irregularities may be a surface finish, sprayed coating, or mechanical or chemical etching. Further, the surface irregularities may be fixed, or may retract and deploy into a position to engage the skeletal structures.

The end cap 30 shown includes cap connectors 34 for coupling the end cap 30 to the central portion 2 of the medical implant 1. The cap connectors 34 shown are round pins to engage the recesses of interface end 22, but in other embodiments are other shapes and include other functions. For example, the cap connectors 34 may be square in cross-section or any other geometric shape. The cap connectors 34 may be oblong for sliding in slots into which they could be engaged, or may have hooked ends to grasp or otherwise capture a portion of the medical implant 1 when coupled. For example, the implant 1 of FIG. 12 includes sliced opening 43 along with other openings for receiving the cap connectors 34. The sliced opening 43 includes a cut 44 that creates a flexible, living hinge capable of securely receiving one of the cap connectors 34. When a cap connector 34 is pushed into the sliced opening 43, the sliced opening 43 deforms to open and allows the cap connector 34 to slide into the sliced opening 43. After the cap connector 34 is seated in the sliced opening 43, the material attempts to return to its pre-insertion position to create a locking effect around the cap connector 34. In addition, or in the alternative, the cap connectors 34 may include relief cuts through some or all of their cross section to provide a living hinge or spring effect when inserted into an appropriately sized opening.

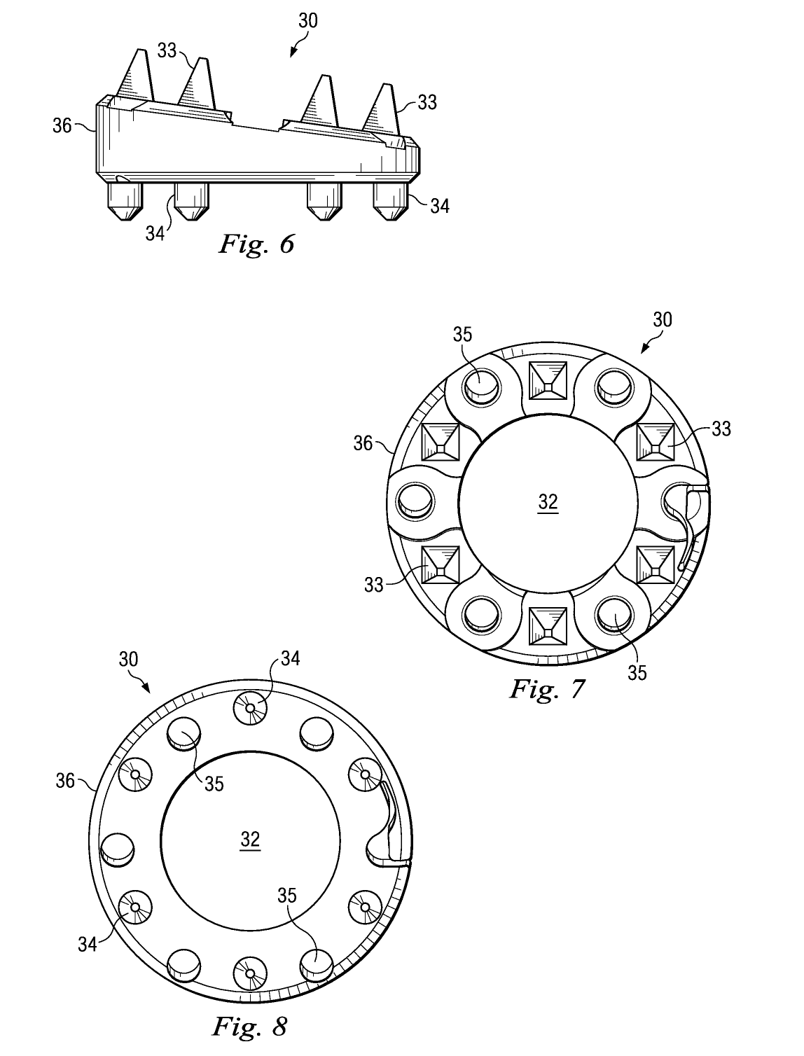

As illustrated in FIG. 5, in some embodiments the end cap 30 has eight equally radially spaced cap connectors 34. This spacing allows for the rotational orientation of the end cap 30 to be altered at forty-five degree increments relative to the tubular members. As illustrated in FIGS. 6-8, in other embodiments the end cap 30 has six radially spaced cap connectors 34, allowing rotational orientation to be altered in sixty degree increments. The adjustable rotational orientations enable implants with end caps of varying thicknesses, such as end cap 30, to be placed from substantially any surgical approach and simultaneously properly match the skeletal structures. For example, to match lordotic or kyphotic spinal angles while approaching from any of anterior, antero-lateral, posterior, postero-lateral, transforaminal, and far lateral approaches. Multiples other than eight may be used in various embodiments, and embodiments with spacing that is not equal may be employed to limit or direct orientation possibilities. The cap connectors 34 illustrated are part of the end cap 30 but in other embodiments, the cap connectors may extend from the central portions 2 of the implant 1 and be connectable to respective openings in the end caps.

As shown, the cap connectors 34 mate with the central portion 2 of the implant 1. In at least one embodiment, the cap connectors 34 snap-fit into the openings of the central portion 2. In some embodiments the cap connectors 34 are adapted for non-destructive or revisable engagement with the central portion. That is, the cap connectors 34 can be disengaged or removed from engagement with the central portion 2 without damaging the central portion or end cap 30. In other embodiments, however, the cap connectors 34 are destructively engaged with the central portion. Examples of types of destructive engagement include, but are not limited to glues, one-way snap-fits, and other engagement mechanisms. In other embodiments, the end cap 30 is molded or sintered to the central portion 2. In addition, in some embodiments the cap connectors 34 and, therefore, the end cap 30 are fixedly secured to the central portion such that no rotation or translation of the end cap 30 relative to the central portion is permitted. In other embodiments, however, the end cap 30 is secured to the central portion 2 in a manner that permits rotational and/or translational movement of the end cap relative to the central portion.

The end cap 30 also includes openings 35 extending through a body 36 of the end cap. Similar to the number of cap connectors 34, the end cap 30 may have varying numbers of openings 35. As shown in FIGS. 5 and 6-8, respectively, the end cap 30 can include eight or six openings 35. Further, the end cap 30 may include any other number of openings 35 or no openings at all. In addition, in some embodiments the openings 35 can be of other shapes and geometries. In yet other embodiments, the openings 35 extend only partially through the body 36 to create recesses.

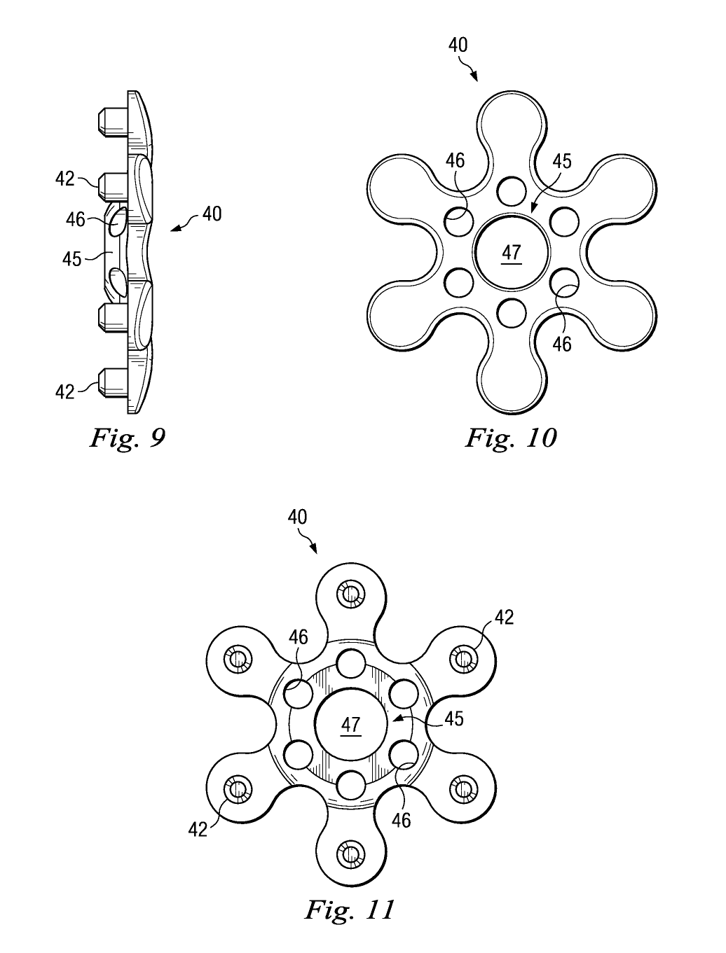

Referring to FIGS. 5 and 9-11, the shoe 40 attaches to the end cap 30 and spans at least a portion of the end cap opening 32. As shown the shoe 40 includes shoe connectors 42 for coupling the shoe 40 to the end cap 30. The shoe connectors 42 shown are round pins, but in other embodiments could be other shapes and could include other functions. For example, the shoe connectors 42 may be square in cross-section or any other geometric shape. The shoe connectors 42 may be oblong for sliding in slots into which they could be engaged, or may have hooked ends to grasp or otherwise capture a portion of the end cap 30 when coupled. The end cap 30 may include sliced openings similar to those described in association with the sliced openings 43 described above. In addition, or in the alternative, the shoe connectors 42 may include relief cuts through some or all of their cross-section to provide a living hinge or spring effect when inserted into an appropriately sized opening.

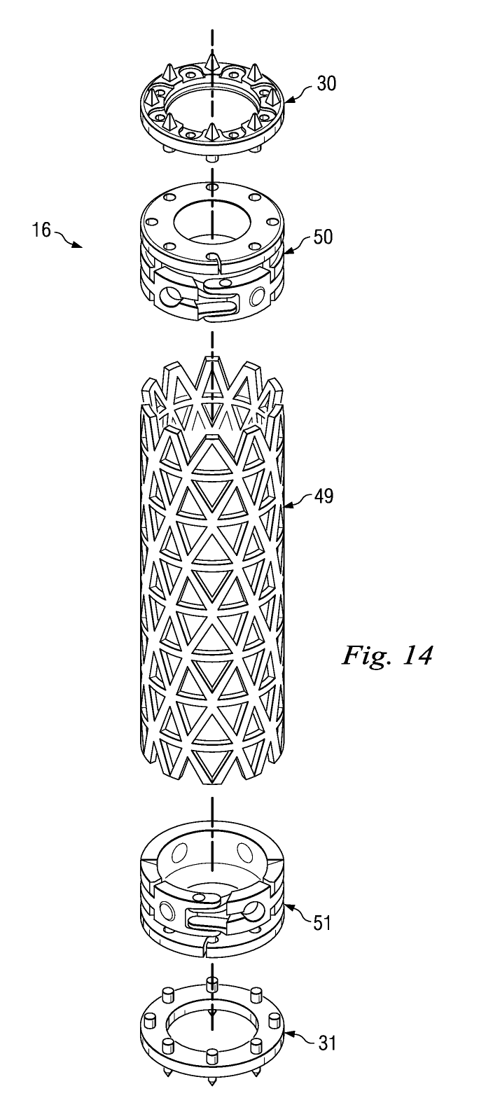

The shoe 40 provides at least in part an interface with the skeletal structure. FIGS. 5 and 9-11 illustrate a shoe 40 that includes a concave shaped recess 45 that extends at least partially into the end cap opening 32. In some embodiments, this configuration may be advantageous because it provides a basket area 45 in the central portion of the shoe 40. The basket area 45 may be useful in receiving a portion of bone growth material that can be held directly against the bone structure, such as an endplate, or may be useful in matching and supporting certain anatomical structures. The shoe 40 also includes a plurality of openings 46 and a central opening 47 to facilitate bone in-growth and fusion. FIGS. 5 and 13 illustrate a shoe 41 that includes a convex shaped portion 48 that extends at least partially away from end cap 31. This shape may be useful for a number of purposes, including matching and supporting adjacent anatomical structures, such as a vertebral endplate. Although the shoe 41 is illustrated as convex, and the shoe 40 is illustrated as concave, note that either shape may be on either end of the medical implant, or only shapes of one type or the other only may be a part of the medical implant 1. Further, shoes of various other shapes such as, but not limited to, flat may also be used.

In some embodiments, the shoes, 40, 41 may be made at least in part from a bioresorbable material. A bioresorbable material provides initial support and an initial containment structure for grafting material that may be placed within the implant. However, over time, the material dissolves and/or the body removes and replaces the material with tissue structures such as bone, thereby providing an especially open pathway through the implant for tissue growth. Examples of bioresorbable materials that could be incorporated in the superior and inferior shoes 40, 41, include but are not limited to allograft, autograft, and xenograft bone materials, polylactide, polyglycolide, tyrosine-derived polycarbonate, polyanhydride, polyorthoester, polyphosphazene, calcium phosphate, hydroxyapatite, bioactive glass, PLLA, PLDA, and combinations thereof.

In other embodiments, the superior and inferior shoes 40, 41 may be at least in part a bioactive substance proportioned to provide a clinical benefit to the recipient of the implant. Bioactive substances include but are not limited to antibiotics or other substances that affect infection, bone growth and bone ingrowth promoting substances, substances that treat or attack cancer cells, or any other substance that makes a therapeutic contribution. Further, the choice of material for shoes 40, 41 can additionally be based upon the desire to use medical diagnostic equipment to monitor fusion or bone in-growth. For example, in some embodiments the shoes 40, 41 (and the end caps 30, 31) are made from a radiolucent material to facilitate the monitoring of bone in-growth via fluoroscopy. Other choices of materials can be selected based on the desire to use other medical imaging or diagnostic equipment and the corresponding effects the materials may or may not have on that imaging choice. For example, in some embodiments the material is a bioresorbable material. Examples of appropriate bioresorbably materials include, but are not limited to allograft, autograft, and xenograft bone materials, polylactide, polyglycolide, tyrosine-derived polycarbonate, polyanhydride, polyorthoester, polyphosphazene, calcium phosphate, hydroxyapatite, bioactive glass, PLLA, PLDA, and combinations thereof.

In some embodiments, the medical implant 1 does not include the shoe 40. In other embodiments, the end cap 30 and the shoe 40 are an integral piece. In yet other embodiments, both the end cap 30 and the shoe 40 interface with the skeletal structure.

Referring now to FIG. 14, an alternative embodiment of an implant 1b is shown. The implant 1b includes a central portion 49, two intermediate end caps 50 and 51, and two engagement end caps 30 and 31. As shown, the end caps 30 and 31 are utilized in combination with end caps 50 and 51 to facilitate monitoring of the fusion with the skeletal structures as described above. In this manner, the end caps 30, 31 may be combined with known implant devices and end caps to facilitate the monitoring of fusion. In that regard, the end caps 30, 31 may be shaped or otherwise configured to mate with various implant devices. In some embodiments, the end caps 30, 31 are utilized in combination with an implant selected from the Sceptor line of implants available from Medtronic, Inc. In some embodiments, the implant 1b utilizes Pyramesh also available from Medtronic, Inc.

In some circumstances, it is advantageous to pack all or a portion of the interior and/or periphery of the implants 1, 1a, and 1b with a suitable osteogenic material, bone morphogenetic proteins, or therapeutic composition. Osteogenic materials include, without limitation, autograft, allograft, xenograft, demineralized bone, synthetic and natural bone graft substitutes, such as bioceramics and polymers, and osteoinductive factors. A separate carrier to hold materials within the device may also be used. These carriers may include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenic compositions may include an effective amount of a bone morphogenetic protein (BMP), transforming growth factor β1, insulin-like growth factor, platelet-derived growth factor, fibroblast growth factor, LIM mineralization protein (LMP), and combinations thereof or other therapeutic or infection resistant agents, separately or held within a suitable carrier material.

Embodiments of the implant in whole or in part may be constructed of biocompatible materials of various types. Examples of implant materials include, but are not limited to, non-reinforced polymers, carbon-reinforced polymer composites, PEEK and PEEK composites, shape-memory alloys, titanium, titanium alloys, cobalt chrome alloys, stainless steel, ceramics and combinations thereof. If the trial instrument or implant is made from radiolucent material, radiographic markers can be located on the trial instrument or implant to provide the ability to monitor and determine radiographically or fluoroscopically the location of the body in the spinal disc space. In some embodiments, the implant or individual components of the implant are constructed of solid sections of bone or other tissues. In other embodiments, the implant is constructed of planks of bone that are assembled into a final configuration. The implant may be constructed of planks of bone that are assembled along horizontal or vertical planes through one or more longitudinal axes of the implant. Tissue materials include, but are not limited to, synthetic or natural autograft, allograft or xenograft, and may be resorbable or non-resorbable in nature. Examples of other tissue materials include, but are not limited to, hard tissues, connective tissues, demineralized bone matrix and combinations thereof. Examples of resorbable materials that may be used include, but are not limited to, polylactide, polyglycolide, tyrosine-derived polycarbonate, polyanhydride, polyorthoester, polyphosphazene, calcium phosphate, hydroxyapatite, bioactive glass, PLLA, PLDA, and combinations thereof. Implant may be solid, porous, spongy, perforated, drilled, and/or open.

FIG. 1 illustrates four vertebrae, V1-V4, of a typical lumbar spine and three spinal discs, D1-D3. While embodiments of the invention may be applied to the lumbar spinal region, embodiments may also be applied to the cervical or thoracic spine or between other skeletal structures.

Other modifications of the present disclosure would be apparent to one skilled in the art. Accordingly, all such modifications and alternatives are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A spinal implant for positioning between a superior vertebra and an inferior vertebra, the device comprising:
a central portion having a superior section, an inferior section, and a longitudinal axis extending therebetween, the central portion made of a first material;
a superior end cap having an inferior surface for engagement with the superior section of the central portion and having a superior surface for engagement with the superior vertebra, the superior end cap made of a second material adapted to facilitate the monitoring of bone in-growth between the superior vertebra and the superior surface, the second material being different than the first material; and
an inferior end cap having an upper surface for engagement with the inferior section of the central portion and having a lower surface for engagement with the inferior vertebra, the inferior end cap made of a third material adapted to facilitate the monitoring of bone in-growth between the inferior vertebra and the lower surface, the third material being different than the first material.

2. The implant of claim 1 wherein the first material is substantially radiopaque and the second and third materials are substantially radiolucent.

3. The implant of claim 2 wherein the first material is a metal and the second material is a polymer.

4. The implant of claim 3 wherein the first material is titanium and the second material is PEEK.

5. The implant of claim 4 wherein the central portion is expandable.

6. The implant of claim 1 wherein the first material substantially reflects energy from an imaging apparatus and the second and third materials are substantially transmissive to the energy from the imaging apparatus.

7. The implant of claim 1 wherein the second and third materials are the same.

8. The implant of claim 6 wherein the superior end cap has a thickness between 0.5 and 10 mm.

9. The implant of claim 8 wherein the thickness of the superior end cap varies so that the superior surface is not perpendicular to the longitudinal axis of the central portion.

10. The implant of claim 9 wherein the inferior end cap has a thickness between 0.5 and 10 mm.

11. The implant of claim 10 wherein the thickness of the inferior end cap varies so that the lower surface is not perpendicular to the longitudinal axis of the central portion.

12. The implant of claim 1 wherein the spinal implant is a corpectomy device.

13. The implant of claim 1 wherein the spinal implant is an interbody device.

14. An expandable medical implant for supporting skeletal structures comprising:
an expandable central portion having a first end section, an opposing second end section, and a longitudinal axis extending therebetween, the central portion formed of a first material that at least partially inhibits the monitoring of bone in-growth using a medical diagnostic technique;
a first end cap having a first mating surface for mating with the first end section of the central portion and having a first engagement surface for engagement with a first portion of the skeletal structure, the first end cap made of a second material different than the first material, the second material inhibiting the monitoring of bone in-growth using the medical diagnostic technique to a lesser degree than the first material; and
a second end cap having a second mating surface for mating with the second end section of the central portion and having a second engagement surface for engagement with a second portion of the skeletal structure, the second end cap made of the second material.

15. The implant of claim 14 wherein the medical diagnostic technique is fluoroscopy.

16. The implant of claim 15 wherein the first material is substantially radiopaque and the second material is substantially radiolucent.

17. The implant of claim 16 wherein the first material is a metal and the second material is a polymer.

18. The implant of claim 17 wherein the first and second end caps each have a thickness between 0.5 and 10 mm.

19. The implant of claim 18 wherein the thicknesses of the first and second caps are non-uniform.

20. The implant of claim 17 wherein the first engagement surface includes a plurality of projections for at least partially penetrating into the first portion of the skeletal structure.

21. The implant of claim 20 wherein the second engagement surface includes a plurality of projections for at least partially penetrating into the second portion of the skeletal structure.

22. An end member for use with a metallic implant for supporting a skeletal structure, the end member comprising:
a body portion having a thickness;
an implant engagement surface extending from the body portion for securely engaging the metallic implant; and
a skeletal engagement surface extending from the body portion opposite the implant engagement surface, the skeletal engagement surface for securely engaging the skeletal structure and promoting bony in-growth between the skeletal structure and the end member;
wherein the end member is formed of a polymer.

23. The member of claim 22 wherein the implant engagement surface includes a plurality of radially spaced projections.

24. The member of claim 23 wherein the skeletal engagement surface includes a plurality of projections for at least partially penetrating the skeletal structure.

25. The member of claim 24 wherein the thickness of the body portion is substantially uniform.

26. The member of claim 24 wherein the thickness of the body portion is variable.

27. The member of claim 26 wherein the variable thickness of the body portion is linear.

28. The member of claim 27 wherein the skeletal structure includes a portion of the spine and the variable linear thickness of the body portion is adapted to substantially match a curvature of the spine.

29. The member of claim 28 wherein the curvature of the spine is lordotic.

30. The member of claim 28 wherein the curvature of the spine is kyphotic.

31. The member of claim 22 wherein the body portion includes a central opening extending therethrough.

32. The member of claim 31 further comprising a shoe for attachment to the end cap, the shoe spanning at least a portion of the central opening and providing at least in part an interface with the skeletal structure

33. The member of claim 32 wherein the shoe includes a concave recess for receiving bone growth material.

34. The member of claim 33 wherein the shoe further includes a plurality of openings to facilitate bony in-growth.

35. The member of claim 34 wherein the shoe is formed from a bioresorbable material.

36. The member of claim 34 wherein the shoe is formed from a polymer.

37. The member of claim 32 wherein the shoe includes a convex projection to substantially match a concave recess of the skeletal structure.

38. The member of claim 22 wherein the skeletal structure is the spine.

39. The member of claim 22 wherein the skeletal structure is a long bone.
